(19) United States
(12) Patent Application Publication
(10) Pub. No.: US 2024/0238003 A1
(43) Pub. Date: Jul. 18, 2024

(54) AORTIC PUNCH

(71) Applicant: The University of Toledo, Toledo, OH (US)

(72) Inventors: Munier Nazzal, Toledo, OH (US); Abdullah Nasif, Toledo, OH (US); Nahidh W. Hasaniya, Toledo, OH (US)

(73) Assignee: The University of Toledo, Toledo, OH (US)

(21) Appl. No.: 18/410,384

(22) Filed: Jan. 11, 2024

Related U.S. Application Data
(60) Provisional application No. 63/438,574, filed on Jan. 12, 2023.

(51) Int. Cl. A61B 17/3205; A61B 17/00

(52) U.S. Cl. CPC A61B 17/32053; A61B 2017/00778; A61B 2017/00982

(57) ABSTRACT

An aortic punch includes a cylindrical barrel, a cylindrical plunger movably mounted within the barrel, a blade shaft movably mounted within a distal end of the barrel and having a first cutting tool at a distal end thereof, the first cutting tool configured to form a first cut in a wall of a blood vessel, and a cylindrical punch body movably mounted within a distal end of the barrel and about the blade shaft and having a second, circular cutting tool configured to form a circular hole in the wall of a blood vessel around the first cut.

### RELATED APPLICATIONS

This application claims priority to U.S. Provisional Application No. 63/438,574, filed under 35 U.S.C. § 111(b) on Jan. 12, 2023, the entire disclosure of which is hereby incorporated by reference for all purposes.

### BACKGROUND OF THE INVENTION

This disclosure relates in general to an aortic punch, and more specifically to an improved structure for such an aortic punch that is configured to open the aorta and punch a hole through the aortic wall in one step.

Known aortic punches are handheld devices that are used to create a circular hole in the aortic wall during coronary artery bypass graft surgery and other aortic procedures, such as a bypass of the mesenteric and renal arteries, and other bypass procedures that originate from large arteries. Patients with coronary artery disease and other arterial diseases often develop this disease over a course of decades. Deposits high in cholesterol, commonly referred to as plaque, builds up in the coronary arteries causing inflammation. The arterial disease tends not to be noticeable until significant blockage or symptoms, such as a heart attack, occurs. A coronary bypass graft surgery is a procedure used to treat coronary artery disease. An aortic punch is a tool used during this procedure to cut a hole accurately and precisely in the wall of the aorta.

Coronary artery disease is the most common heart disease type as well as the leading cause of death in both women and men in the United States, and coronary bypass graft surgery is one of the main treatments for coronary artery disease. There are more than 200,000 coronary bypass graft surgery procedures performed each year in the US.

However, coronary bypass graft surgery can be very intense as during the procedure the patient's heart is stopped temporarily and they are put on heart-lung bypass machine. It has been shown that more complications arise in patients that spend more time on the bypass machine. Also, because the surgery is not actually a cure for the underlining heart disease, there will be need of medications after the procedure and between 20 to 30 percent of patients will need a second procedure within 10 years. Part of the procedure is to connect a vein or an artery to the aorta which is a large blood vessel. Connecting a vein or an artery to the aorta requires opening a small incision in the aorta using a knife then enlarged and formed into a hole using another device, a puncher, in a two-step process—first making an incision, and then punching a hole.

The known two-step process used to open the aorta in preparation for anastomosis is time-consuming and may be complicated by dissection if the puncher is not negotiated through the proper plane. In addition, because two separate devices are used to create the desired round hole, there is a risk of different cut and punch diameters that results in a malformed punch geometry, for example undesirable notches N, as shown in FIG. 1, may be formed that extend radially outward from the periphery of the punched round hole H punched in the aorta A, and may require stiches to repair. Additionally, during such a known two-step process, the puncher must be negotiated through a 3-layer diseased aorta wall that may result in creating a flap or dissection, especially in patients with calcification and plaques, which can compromise the wall of the artery and at least require repair of the wall before proceeding with the remainder of the procedure. Problematically, if the dissection is not noticed, it may cause complications of dissection and extension of the dissection after the procedure.

Thus, it would be desirable to provide an improved structure for an aortic punch that is configured to open the aorta and punch a hole through the aorta wall in one step. This avoids exchange of instruments and allows the puncher to be introduced in the same hole as the cutting part.

### SUMMARY OF THE INVENTION

The present disclosure relates to an improved structure for an aortic punch that is configured to open the aorta and punch a hole through the aorta wall in one step. The aortic punch includes a cylindrical barrel, a cylindrical plunger movably mounted within the barrel, a blade shaft movably mounted within a distal end of the barrel and having a first cutting tool at a distal end thereof, the first cutting tool configured to form a first cut in a wall of a blood vessel, and a cylindrical punch body movably mounted within a distal end of the barrel and about the blade shaft and having a second, circular cutting tool configured to form a circular hole in the wall of a blood vessel around the first cut.

A method of forming a circular hole in a wall of an aorta includes providing an aortic punch including a cylindrical barrel, a cylindrical plunger movably mounted within the barrel, a blade shaft movably mounted within a distal end of the barrel and having a first cutting tool at a distal end thereof, the first cutting tool configured to form a first cut in a blade shaft movably mounted within a distal end of the barrel and having a first cutting tool at a distal end thereof, and a cylindrical punch body movably mounted within a distal end of the barrel and about the blade shaft and having a second, circular cutting tool configured to form a circular hole in the wall of a blood vessel around the first cut. A first opening is formed in the wall of the aorta with the first cutting tool, and a second opening is formed in the wall of the aorta with the circular cutting tool in a one-step process.

Various aspects of the present disclosure will become apparent to those skilled in the art from the following detailed description of the preferred embodiment, when read in view of the accompanying drawings.

### BRIEF DESCRIPTION OF THE DRAWINGS

FIG. 1 is a plan view of a hole formed in an artery using a scalpel and a conventional aortic punch, showing notches extending radially outward from the periphery of the hole.

FIG. 2 is a perspective view of an aortic punch in accordance with the present disclosure.

FIG. 3 is an exploded perspective view of the aortic punch shown in FIG. 2.

FIG. 4 is an enlarged perspective view of the punch body and the blade shaft shown in FIG. 3.

FIG. 5A is a perspective view of a portion of the aortic punch shown in FIGS. 2 and 3.

FIG. 5B is an end view of the aortic punch shown in FIGS. 2, 3, and 5A.

FIG. 6A is a perspective view of a portion of an alternate embodiment of the aortic punch shown in FIGS. 2 and 3.

FIG. 6B is an end view of the aortic punch shown in FIG. 6A.

FIG. 7A is a plan view of an alternate embodiment of the blade shaft shown in FIG. 3.

FIG. 7B is a perspective view of the embodiment of the blade shaft shown in FIG. 7A.

FIG. 8 is an enlarged plan view of the aortic punch shown in FIGS. 2 and 3.

FIG. 9A is an enlarged perspective view of the aortic punch shown in FIGS. 2, 3, and 8, showing a first step in the formation of a hole in an aorta.

FIG. 9B is an enlarged perspective view of the aortic punch shown in FIGS. 2, 3, and 8, showing a second step in the formation of a hole in an aorta.

FIG. 9C is an enlarged perspective view of the aortic punch shown in FIGS. 2, 3, and 8, showing a third step in the formation of a hole in an aorta.

FIG. 9D is an enlarged perspective view of the aortic punch shown in FIGS. 2, 3, and 8, showing a fourth step in the formation of a hole in an aorta.

FIG. 9E is an enlarged perspective view of the aortic punch shown in FIGS. 2, 3, and 8, showing a fifth step in the formation of a hole in an aorta.

FIG. 9F is an enlarged perspective view of the aortic punch shown in FIGS. 2, 3, and 8, showing a sixth step in the formation of a hole in an aorta.

### DETAILED DESCRIPTION

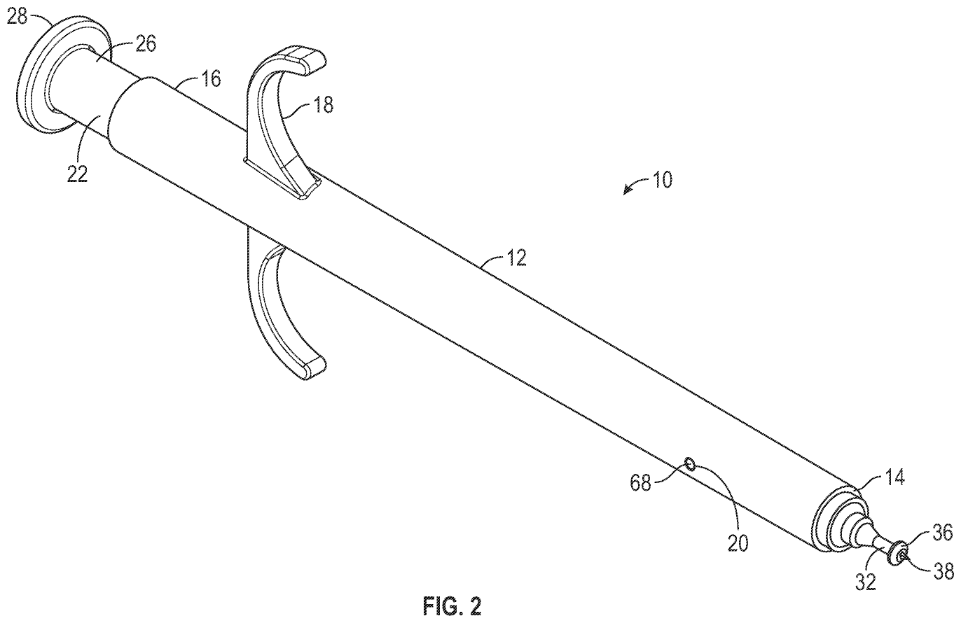

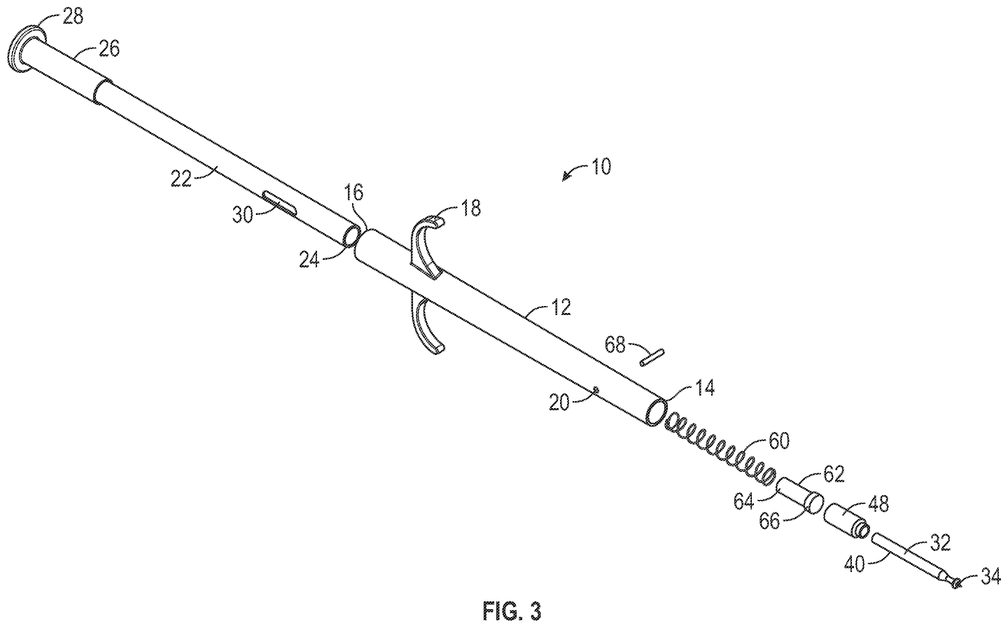

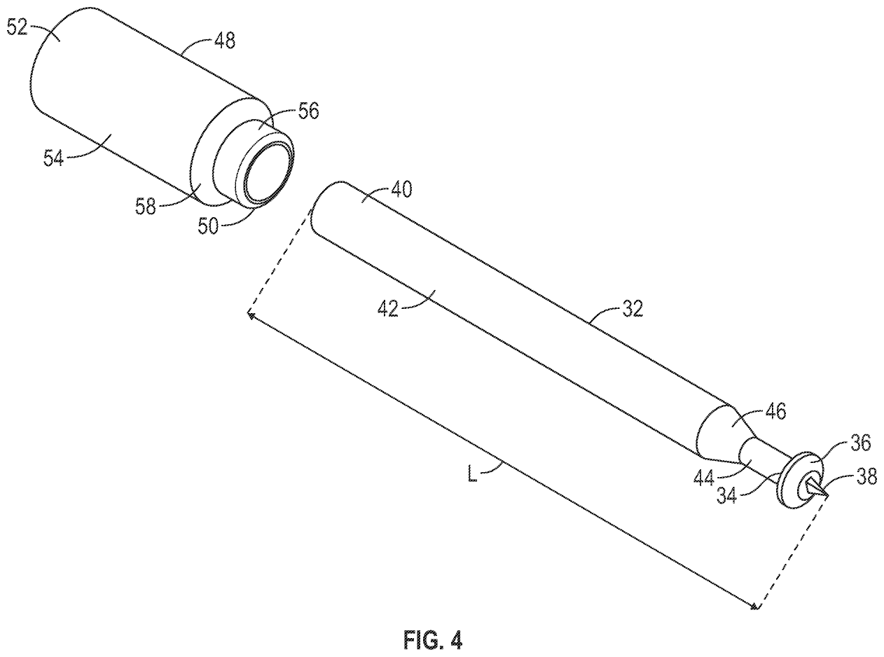

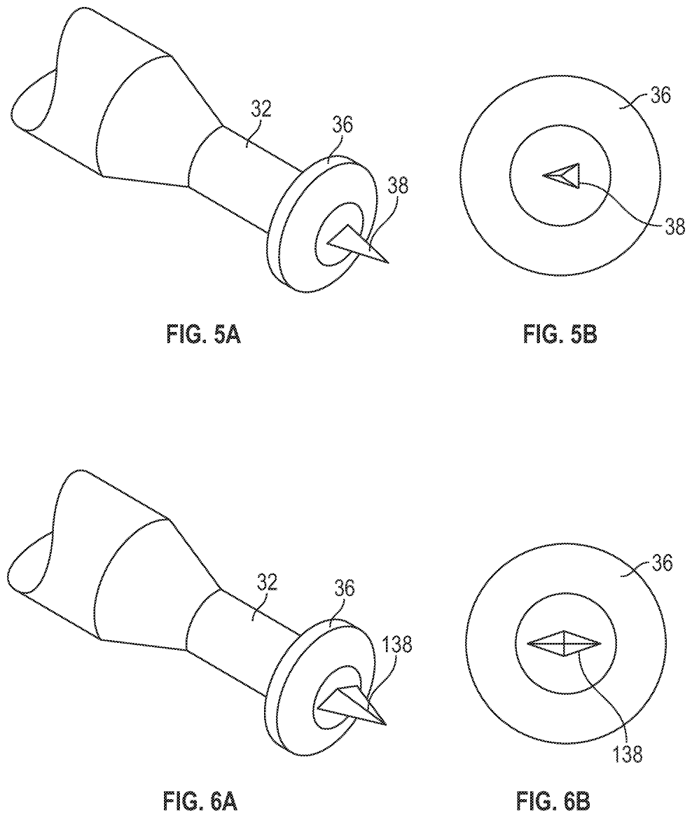

Referring now to the drawings, there is illustrated in FIGS. 2 through 6B a basic structure of an aortic punch, shown generally at 10. Although the aortic punch 10 is described herein as being used during coronary procedures, it will be understood that the embodiments of the aortic punch 10 described herein may be used during any bypass procedures originating from the aorta or other large arteries.

The illustrated aortic punch 10 includes an elongated, cylindrical body or barrel 12 defining a longitudinally extending opening, and having a first end 14, a second end 16, a pair of arcuate finger holds 18, and opposing pin holes 20 formed through a wall of the barrel 12.

An elongated, cylindrical plunger 22 has a first end 24, a second end 26 having a plunger flange 28 formed thereon, and opposing, longitudinally extending pin slots 30 formed through a wall of the plunger 22. The plunger 22 has an outside diameter smaller than an inside diameter of the barrel 12 and is configured for slidable mounting within the barrel 12.

An elongated, cylindrical blade shaft 32 has a first end 34 having a blade flange 36 formed thereon, a first cutting tool or blade 38 extending longitudinally outwardly from the blade flange 36, and a second end 40. The blade shaft 32 has a first portion 42 having a first diameter, and a second portion 44 adjacent the blade flange 36 having a second diameter smaller than the first diameter. A tapered portion 46 extends between the first portion 42 and the second portion 44.

Although the illustrated blade shaft 32 has two diameters, it will be understood that the blade shaft 32 may be formed with one or more than two diameters. The illustrated blade shaft 32 may have any desired length L, such as within the range of about 10 mm to about 20 mm. It will be understood that the length of the blade shaft 32 may vary based on the size and condition of the aorta or other blood vessels through which a hole will be formed. Additionally, the aortic punch 10 may be configured such that the blade shafts of different lengths and having different blades, described below, may be interchangeably used with the aortic punch 10.

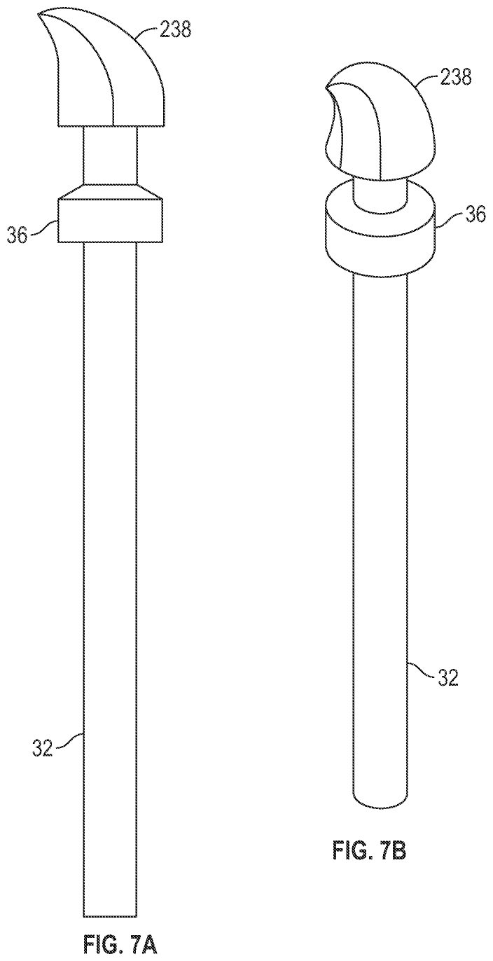

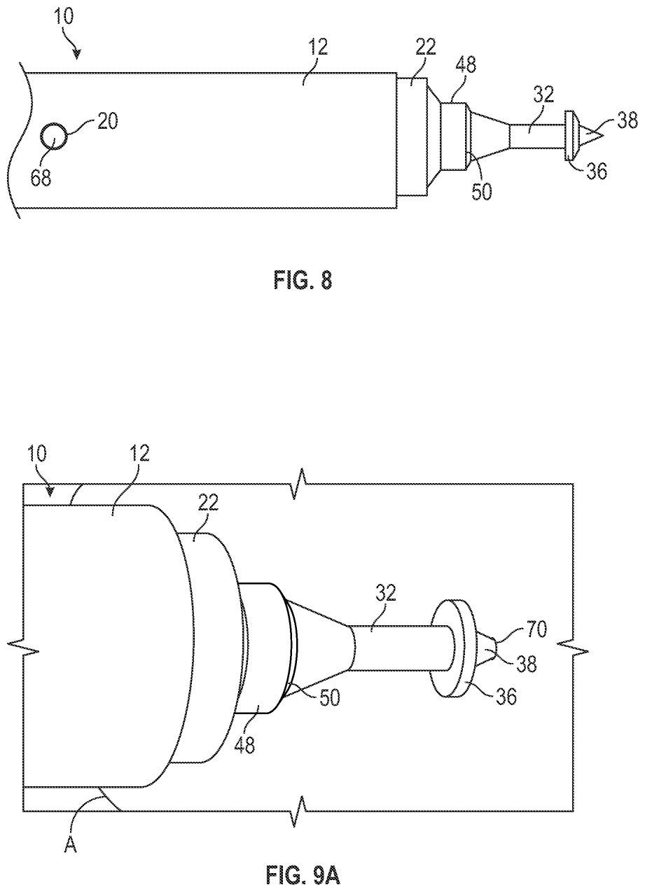

The first cutting blade 38, as shown in FIGS. 2 through 5B, has a triangular base and three triangular faces defining three cutting edges, one between each adjacent face. Like the blade shaft 32, the size and shape of the blade 38 may vary based on the size and condition of the aorta or other blood vessels through which a hole will be formed. As shown in FIGS. 6A and 6B for example, the blade 138 may have a diamond shaped base and four triangular faces defining four cutting edges, one between each adjacent face. Additionally, as shown in FIGS. 7A and 7B, the blade 238 may be rhino-shaped.

A cylindrical punch body 48 has a first end 50, a second end 52, a first portion 54 having first diameter, and a second portion 56 having a second diameter smaller than the first diameter. A tapered portion 58 extends between the first portion 54 and the second portion 56. The circular first or distal end 50 of the punch body 48 defines a second or circular cutting tool or edge.

A coil spring 60 extends between the first end 24 of the plunger 22 and the second end 52 of the punch body 48. A punch engagement member 62 has a cylindrical body 64 having a first diameter and circular flange 66 formed on a first end thereof. The cylindrical body 64 is configured to be inserted into a first end of the coil spring 60 such that the flange 66 engages the second end 52 of the punch body 48. A locking pin 68 extends through the pin holes 20 of the barrel 12 and through the pin slots 30 formed in the plunger 22.

The illustrated aortic punch 10 combines the conventional steps of forming a first opening the aorta (or other blood vessels) and then punching a second opening or circular hole into a one-step process. Advantageously, the aortic punch 10, and the other embodiments of the aortic punch illustrated and described herein, may be used on blood vessels of different sizes.

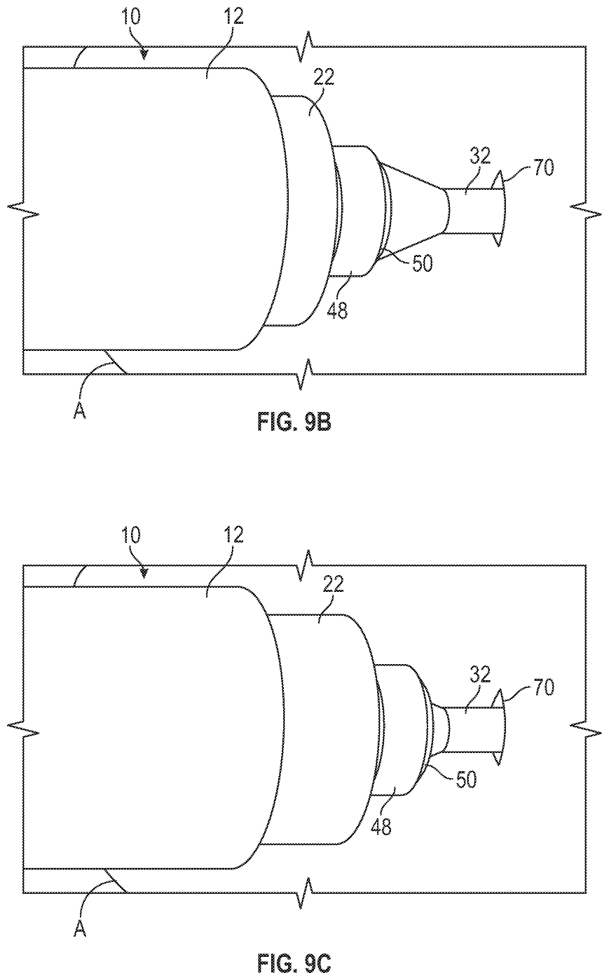

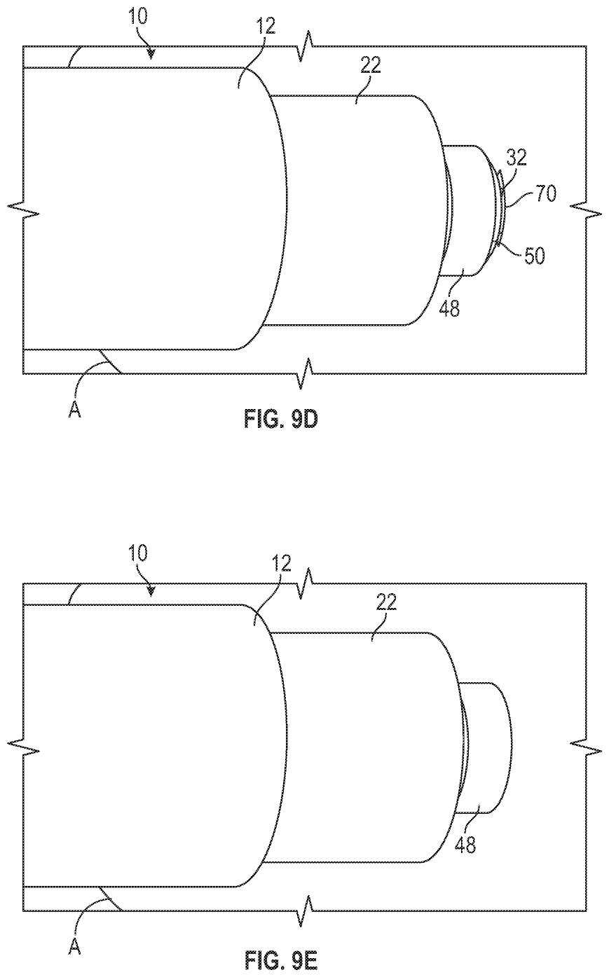

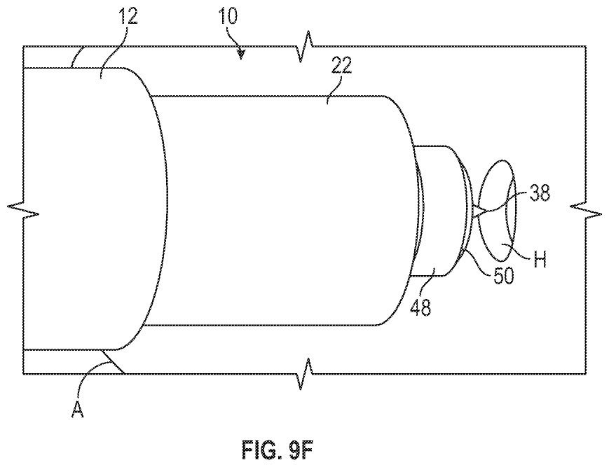

FIGS. 9A through 9E show the various steps used to punch a hole in a blood vessel, such as an aorta A when using the aortic punch 10. FIG. 9A illustrates a first step in the process of punching a hole H in the aorta A wherein the blade 38 is used to create or cut an incision 70 in the aortic wall. In the first step, the surgeon pushes the blade through the aortic wall until the incision is formed.

As shown in FIGS. 9B and 9C, after the incision 70 is cut by the blade 38, the distal end of the blade shaft 32 is pushed through the incision 70 in the aortic wall. In this step, the surgeon pushes on the plunger 22, thus urging the circular cutting edge of the distal end 50 of the punch body 48 into contact with the aortic wall.

As shown in FIGS. 9D and 9E, the surgeon continues to push the plunger 22 longitudinally forward; i.e., toward the aortic wall, thus urging the circular cutting edge of the distal end 50 of the punch body 48 through the aortic wall, thus forming the hole H. The surgeon then removes the aortic punch 10 from the newly formed hole H, as shown in FIG. 9F.

Advantageously, the aortic punch 10 eliminates steps needed to create a hole H in the aorta or other blood vessel when using a conventional aortic punch, thus making coronary artery bypass graft surgery more stream-lined and efficient. The process of creating the hole H with the aortic punch 10 is smoother and does not require negotiating a previously cut incision in the artery with a conventional aortic punch and creating a risk for dissection.

Additional advantages are achieved by combining a knife blade, such as the blade 38, and a punching tool, such as the cutting edge 50, into one device, including: an operating room will require one fewer surgical instrument during coronary artery bypass graft surgery; the coronary artery bypass graft surgery will require fewer steps; the potential for injury to the surgeon or the staff is reduced; and the coronary artery bypass graft surgery procedure is more efficient that if using the conventional method, i.e., using a knife or scalpel and a conventional aortic punch.

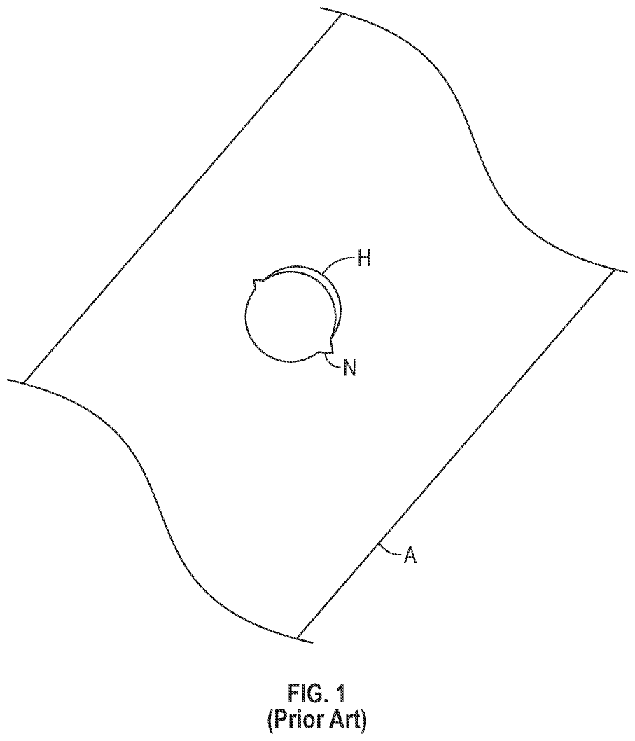

The combination of the knife blade and punching tool into one device, the aortic punch 10, will also eliminate the risk of incorrect hole geometry. For example, the use of two separate devices, a scalpel and a conventional aortic punch, is known to result in the initial cut and the subsequent punch to have slightly different sizes or diameters, leading to malformed puncture geometry, for example as shown in FIG. 1. Forming punched holes with the aortic punch 10 significantly reduces this risk.

The principle and mode of operation of the invention have been explained and illustrated in its preferred embodiment. However, it must be understood that the invention may be practiced otherwise than as specifically explained and illustrated without departing from its spirit or scope.

What is claimed is:

1. An aortic punch comprising:
a cylindrical barrel;
a cylindrical plunger movably mounted within the barrel;
a blade shaft movably mounted within a distal end of the barrel and having a first cutting tool at a distal end thereof, the first cutting tool configured to form a first cut in a wall of a blood vessel; and
a cylindrical punch body movably mounted within a distal end of the barrel and about the blade shaft and having a second, circular cutting tool configured to form a circular hole in the wall of a blood vessel around the first cut.

2. The aortic punch according to claim 1, wherein the first cutting tool is a blade having a triangular base and three triangular faces defining three cutting edges, one cutting edge between each adjacent face.

3. The aortic punch according to claim 1, wherein the first cutting tool is a blade having a diamond shaped base and four triangular faces defining four cutting edges, one cutting edge between each adjacent face.

4. The aortic punch according to claim 1, wherein the first cutting tool is a blade that is rhino-shaped.

5. The aortic punch according to claim 2, wherein the barrel includes opposing pin holes formed through a wall thereof.

6. The aortic punch according to claim 5, wherein the cylindrical punch body has a first end, a second end, a first portion having a first diameter, and a second portion having a second diameter smaller than the first diameter, wherein a tapered portion extends between the first portion and the second portion, and wherein an edge of the circular distal end of the punch body defines the circular cutting tool.

7. The aortic punch according to claim 6, further including a coil spring extending between a first end of the plunger and the second end of the punch body.

8. The aortic punch according to claim 7, further including a punch engagement member having a cylindrical body and circular flange formed on a first end thereof, wherein the cylindrical body is configured to be inserted into a first end of the coil spring such that the flange engages the second end of the punch body.

9. The aortic punch according to claim 8, wherein the cylindrical plunger has a first end, a second end having a plunger flange formed thereon, and opposing, longitudinally extending pin slots formed through a wall of the plunger, wherein the plunger has an outside diameter smaller than an inside diameter of the barrel and is configured for slidable mounting within the barrel.

10. The aortic punch according to claim 9, further including a locking pin extending through the pin holes of the barrel and through the pin slots formed in the plunger.

11. A method of forming a circular hole in a wall of a blood vessel, the method comprising:
providing an aortic punch including:
a cylindrical barrel;
a cylindrical plunger movably mounted within the barrel;
a blade shaft movably mounted within a distal end of the barrel and having a first cutting tool at a distal end thereof, the first cutting tool being configured to form a first cut in a blade shaft movably mounted within a distal end of the barrel and having a first cutting tool at a distal end thereof; and
a cylindrical punch body movably mounted within a distal end of the barrel and about the blade shaft and having a second, circular cutting tool configured to form a circular hole in the wall of a blood vessel around the first cut;
forming a first opening in the wall of the blood vessel with the first cutting tool; and
forming a second opening in the wall of the blood vessel with the circular cutting tool in a one-step process.

12. The method according to claim 11, wherein the first cutting tool is a blade having a triangular base and three triangular faces defining three cutting edges, one cutting edge between each adjacent face.

13. The method according to claim 11, wherein the first cutting tool is a blade having a diamond shaped base and four triangular faces defining four cutting edges, one cutting edge between each adjacent face.

14. The method according to claim 11, wherein the first cutting tool is a blade that is rhino-shaped.

15. The method according to claim 12, wherein the first opening formed by the blade is an incision.

16. The method according to claim 15, wherein the circular cutting tool forms a circular hole in the wall of the blood vessel around the incision.

17. The method according to claim 16, wherein the cylindrical punch body has a first end, a second end, a first portion having first diameter, and a second portion having a second diameter smaller than the first diameter, wherein a tapered portion extends between the first portion and the second portion, and wherein an edge of the circular distal end of the punch body defines the circular cutting tool.

18. The method according to claim 17, wherein the step of forming the first opening in the wall of the blood vessel with the first cutting tool includes urging the blade, mounted to the distal end of the blade shaft, through the wall of the blood vessel to form the incision therein.

19. The method according to claim 18, wherein the step of forming the second opening in the wall of the blood vessel with the circular cutting tool in a one-step process includes urging the circular cutting tool through the wall of the blood vessel around the incision while immediately after forming the incision.

20. The method according to claim 19, wherein the circular cutting tool forms a circular hole in the wall of the blood vessel around the incision.

21. The method according to claim 11, wherein the blood vessel is an aorta.
